(19) United States
(12) Patent Application Publication
(10) Pub. No.: US 2011/0118118 A1
(43) Pub. Date: May 19, 2011

(54) HERBICIDALLY ACTIVE PHENYL-SUBSTITUTED PYRIDAZINONES

(75) Inventors: Stefan Lehr, Liederbach (DE); Jan Dittgen, Gent (BE); Christopher Hugh Rosinger, Hofheim (DE); Dieter Feucht, Eschborn (DE); Isolde Häuser-Hahn, Leverkusen (DE)

(73) Assignee: BAYER CROPSCIENCE AG, MONHEIM (DE)

(21) Appl. No.: 12/888,489

(22) Filed: Sep. 23, 2010

Related U.S. Application Data
(60) Provisional application No. 61/246,311, filed on Sep. 28, 2009.

(30) Foreign Application Priority Data
Sep. 25, 2009 (EP) 09012171.6

(51) Int. Cl. A01N 25/32; C07D 237/16; A01N 43/58; A01P 3/00; A01P 7/00; A01P 13/00; A01P 21/00

(52) U.S. Cl. 504/103; 544/240; 504/238; 504/137

(57) ABSTRACT

Phenyl-substituted pyridazinones of the formula (I) are described as herbicides and insecticides.

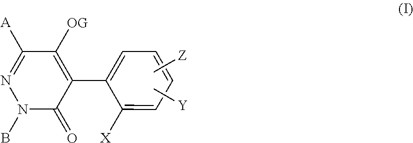

In this formula (I), A, B, G, X, Y and Z represent radicals such as hydrogen, organic radicals such as alkyl, and other radicals such as halogen, nitro and cyano.

### CROSS REFERENCE TO RELATED APPLICATIONS

This application claims priority to European Application 0902171.6 filed Sep. 25, 2009 and U.S. Application 61/246,311 filed Sep. 28, 2009. The entire contents of which are hereby incorporated by reference in their entireties.

### BACKGROUND OF THE INVENTION

1. Field of the Invention

The invention relates to the technical field of crop protection agents, in particular that of herbicides for the selective control of broad-leaved weeds and weed grasses in crops of useful plants.

2. Description of Related Art

Specifically, it relates to aryl-substituted pyridazinone derivatives, to processes for their preparation and to their use as herbicides.

Various publications describe substituted 4-phenylpyridazinones having herbicidal properties. 2-Methyl-4-phenylpyridazinones are known from Stevenson et. al, J. Het. Chem., (2005), 427 ff. WO2007/119434 A1 mentions 4-phenylpyridazinones carrying an alkyl radical in the 2-position of the phenyl ring. WO2009/035150 describes 4-phenylpyridazinones which are 2-,4-dichlorosubstituted at the phenyl ring.

The herbicidal activity of the compounds known from these publications, however, is frequently inadequate. Accordingly, it is an object of the present invention to provide alternative herbicidally active compounds.

### SUMMARY

It has been found that 4-phenylpyridazinones whose phenyl ring carries certain substituents are particularly suitable as herbicides.

The present invention provides 4-phenylpyridazinones of the formula (I) or salts thereof

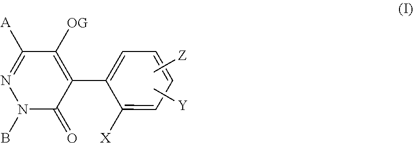

in which
A is hydrogen or (C₁-C₆)-alkyl;
B is hydrogen, (C₁-C₆)-alkyl or (C₁-C₆-alkoxy)-C₁-C₆-alkyl;
n is 0, 1, 2 or 3;
G is hydrogen, C(═O)R¹, C(=L)MR², SO₂R³, P(=L)R⁴R⁵, C(=L)NR⁶R⁷ or E;
E is a metal ion equivalent or an ammonium ion;
L is oxygen or sulfur;
M is oxygen or sulfur;
R¹ is (C₁-C₆)-alkyl, (C₂-C₆)-alkenyl, (C₁-C₄)-alkoxy-(C₁-C₆)-alkyl, di-(C₁-C₄)-alkoxy-(C₁-C₆)-alkyl or (C₁-C₄)-alkylthio-(C₁-C₆)-alkyl, each of which is substituted by n halogen atoms,
a fully saturated 3- to 6-membered ring consisting of 3 to 5 carbon atoms and 1 to 3 heteroatoms from the group consisting of oxygen, sulfur and nitrogen, which ring is substituted by n radicals from the group consisting of halogen, (C₁-C₄)-alkyl and (C₁-C₄)-alkoxy,
phenyl, phenyl-(C₁-C₄)-alkyl, heteroaryl, phenoxy-(C₁-C₄)-alkyl, heteroaryloxy-(C₁-C₄)-alkyl or (C₃-C₆)-cycloalkyl substituted by n radicals from the group consisting of halogen, (C₁-C₄)-alkyl and (C₁-C₄)-alkoxy;
R² is (C₁-C₆)-alkyl, (C₂-C₆)-alkenyl, (C₁-C₄)-alkoxy-(C₁-C₆)-alkyl or di-(C₁-C₄)-alkoxy-(C₁-C₆)-alkyl, each of which is substituted by n halogen atoms,
or (C₃-C₆)-cycloalkyl, phenyl or benzyl, each of which is substituted by n radicals from the group consisting of halogen, (C₁-C₄)-alkyl and (C₁-C₄)-alkoxy;
R³, R⁴ and R⁵ are independently of one another (C₁-C₆)-alkyl, (C₁-C₄)-alkoxy, N—(C₁-C₆)-alkylamino, N,N-di-(C₁-C₆)-alkylamino, (C₁-C₄)-alkylthio, (C₂-C₄)-alkenyl or (C₃-C₆)-cycloalkylthio, each of which is substituted by n halogen atoms,
or benzyl, phenoxy, phenylthio or phenyl substituted by n radicals from the group consisting of halogen, (C₁-C₄)-alkyl and (C₁-C₄)-alkoxy;

R⁶ and R⁷ independently of one another are each hydrogen,
(C₃-C₆)-cycloalkyl, (C₂-C₆)-alkenyl, (C₁-C₆)-alkoxy, (C₁-C₄)-alkoxy-(C₁-C₆)-alkyl or (C₁-C₆)-alkyl substituted by n halogen atoms,
phenyl or benzyl, each of which is substituted by n radicals from the group consisting of halogen, (C₁-C₄)-alkyl and (C₁-C₄)-alkoxy;
or R⁶ and R⁷ together with the nitrogen atom to which they are attached form a 3- to 6-membered ring comprising 2 to 5 carbon atoms and 0 or 1 oxygen or sulfur atoms;
X is (C₁-C₆)-alkyl or (C₁-C₆)-alkyloxy;
Y is halogen;
Z is halogen,
with the proviso that Y and Z are not both chlorine located in the 2- and 4-position.

### DETAILED DESCRIPTION OF A PREFERRED EMBODIMENT

If G and/or B are/is hydrogen, the compounds of the formula (I) according to the invention can, depending on external conditions such as pH, solvent and temperature, be present in various tautomeric structures, all of which are embraced by the formula (I):

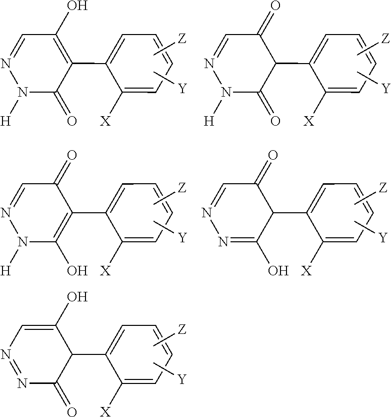

In all the structures below, the substituents, unless defined otherwise, have the same meaning as given above for compounds of the formula (I).

Compounds of the formula (I) according to the invention in which G is hydrogen can be prepared, for example, in accordance with the method shown in Scheme 1 by a base-induced condensation reaction of compounds of the formula (II). Here, R9 is (C1-C6)-alkyl, in particular methyl or ethyl. This method is also known, for example, from EP 2 042 491 A1.

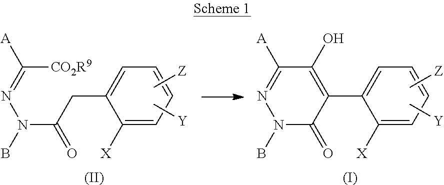

Compounds of the formula (II) can be prepared, for example, in accordance with the method shown in Scheme 1a by reaction of hydrazonocarboxylic acid derivatives with phenylacetic acid derivatives. Here, U is a leaving group introduced by carboxylic acid-activating reagents such as carbonyldiimidazole, carbonyldiimides (such as, for example, dicyclohexylcarbodiimide), phosphorylating agents (such as, for example, POCl₃, BOP—Cl), halogenating agents such as, for example, thionyl chloride, oxalyl chloride, phosgene or chloroformic esters. Such methods are also known to the person skilled in the art from WO2007/119434 and documents cited therein.

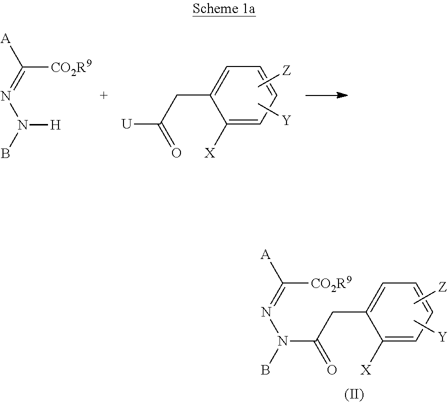

Compounds of the formula (II) can also be prepared, for example, according to the method shown in Scheme 1b by the reaction known to the person skilled in the art from Zh. Obs. Khim. 1992, 62, 2262 of hydrazides (IIa) with ketocarboxylic acids of the formula A-CO—CO₂R⁹.

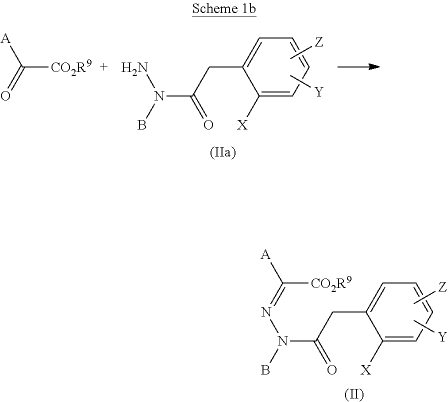

The hydrazides of the formula (IIa) mentioned in Scheme 1 b can be prepared, for example, by reaction of hydrazines of the formula B—NH—NH₂ with the phenylacetic acid derivatives shown in Scheme 1a, according to the method described in J. Org. Chem. 1980, 45, 3673. The hydrazides shown in Scheme 1a can be prepared from the ketocarboxylic acids A-CO—CO₂R⁹ shown in Scheme 1 b and known per se, for example according to the methods described in J. Med. Chem. 1985 (28), 1436.

The free phenylacetic acids required for preparing the phenylacetic acid derivatives shown in Scheme 1a, i.e. those in which U is hydroxyl, are known or can be prepared by processes known per se and, for example, from WO 2005/075401, WO 2001/96277, WO 1996/35664 and WO 1996/25395.

However, certain phenylacetic acid derivatives can also be prepared using acetate enolates in the presence of palladium catalysts formed, for example, from a palladium source (for example Pd₂(dba)₃ or Pd(OAc)₂) and a ligand (for example (t-Bu)₃P, iMes*HCl or 2′-(N,N-dimethylamino)-2-(dicyclohexylphosphanyl)biphenyl) (WO 2005/048710, J. Am. Chem. Soc 2002. 124, 12557, J. Am. Chem. Soc 2003. 125, 11176 or J. Am. Chem. Soc. 2001, 123, 799). In addition, certain substituted aryl halides can be converted under copper catalysis into the corresponding substituted malonic esters (described, for example, in Org. Lett. 2002, 2, 269, WO 2004/108727), which can be converted by known methods into phenylacetic acids.

Compounds of the formula (I) according to the invention in which G is hydrogen can also be prepared, for example, according to the method shown in Scheme 2 by reaction of compounds of the formula (I) in which G is R⁸ with strong mineral bases such as sodium hydroxide or potassium hydroxide, or in concentrated mineral acids such as hydrobromic acid.

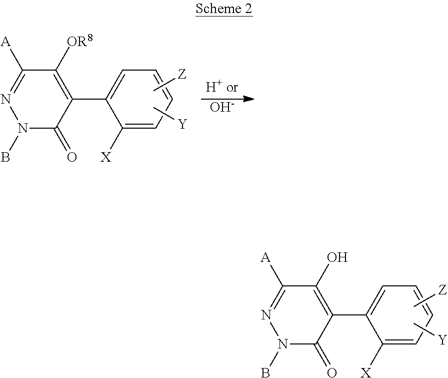

Compounds of the formula (I) according to the invention in which G is C(═O)R¹ can be prepared, for example, by reactions known to the person skilled in the art of compounds of the formula (I) in which G is hydrogen with carbonyl halides of the formula Hal-CO—R¹ or with carboxylic anhydrides of the formula R¹—CO—O—CO—R¹.

Compounds of the formula (I) according to the invention in which G is C(=L)MR² can be prepared, for example, by reactions known to the person skilled in the art of compounds of the formula (I) in which G is hydrogen with a) chloroformic esters or chloroformic thioesters of the formula R²-M-COOR¹ or b) with chloroformyl halides or chlorothioformyl halides.

Compounds of the formula (I) according to the invention in which G is SO₂R³ can be prepared, for example, by reactions known to the person skilled in the art of compounds of the formula (I) in which G is hydrogen with sulfonyl chlorides of the formula R³—SO₂—Cl.

Compounds of the formula (I) according to the invention in which G is P(=L)R⁴R⁵ can be prepared, for example, by reactions known to the person skilled in the art of compounds of the formula (I) in which G is hydrogen with phosphoryl chlorides of the formula Hal-P(=L)R⁴R⁵.

Compounds of the formula (I) according to the invention in which G is E can be prepared, for example, by reactions known to the person skilled in the art of compounds of the formula (I) in which G is hydrogen with metal compounds of the formula Me(OR10)t or with amines. Here, Me is a monovalent or divalent metal ion, preferably an alkali metal or alkaline earth metal such as lithium, sodium, potassium, magnesium or calcium. The index t is 1 or 2. An ammonium ion is the group NH4+ or R13R14R15R16N+ in which R13, R14, R15 and R16 independently of one another are preferably (C1-C6)-alkyl or benzyl.

Compounds of the formula (I) according to the invention in which G is C(=L)NR6R7 can be prepared, for example, by reactions known to the person skilled in the art of compounds of the formula (I) in which G is hydrogen with isocyanates or isothiocyanates of the formula R⁶—N═C=L or with carbamoyl chlorides or thiocarbamoyl chlorides of the formula R⁶R⁷N—C(=L)Cl.

Depending on the nature of the substituents defined above, the compounds of the formula (I) have acidic or basic properties and can form salts, if appropriate also inner salts, or adducts with inorganic or organic acids or with bases or with metal ions. If the compounds of the formula (I) carry amino, alkylamino or other groups which induce basic properties, these compounds can be reacted with acids to give salts, or they are directly obtained as salts in the synthesis. Examples of inorganic acids are hydrohalic acids, such as hydrogen fluoride, hydrogen chloride, hydrogen bromide and hydrogen iodide, sulfuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulfonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminum, tin and lead, and also of the first to eighth transition group, in particular chromium, manganese, iron, cobalt, nickel, copper, zinc and others. Particular preference is given to the metal ions of the elements of the fourth period. Here, the metals can be present in the various valencies that they can assume. If the compounds of the formula (I) carry hydroxyl, carboxyl or other groups which induce acidic properties, these compounds can be reacted with bases to give salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl groups, mono-, di- and trialkanolamines of (C₁-C₄)-alkanols, choline and also chlorocholine.

Halogen is fluorine, chlorine, bromine or iodine.

A metal ion equivalent is a metal ion with one positive charge, such as Na⁺, K⁺, (Mg²⁺)_{1/2}, (Ca²⁺)_{1/2}, MgH⁺, CaH⁺, (Al³⁺)_{1/3}, (Fe²⁺)_{1/2} or (Fe³⁺)_{1/3}.

Alkyl means saturated, straight-chain or branched hydrocarbon radicals having 1 to 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Haloalkyl means straight-chain or branched alkyl groups having 1 to 8 carbon atoms (as mentioned above), where in these groups some or all of the hydrogen atoms may be replaced by halogen atoms, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.

Alkenyl means unsaturated, straight-chain or branched hydrocarbon radicals having 2 to 8 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkoxy means saturated, straight-chain or branched alkoxy radicals having 1 to 8 carbon atoms, for example C1-C6-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-di-methylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;

haloalkoxy means straight-chain or branched alkoxy groups having 1 to 8 carbon atoms (as mentioned above), where in these groups some or all of the hydrogen atoms may be replaced by halogen atoms as mentioned above, for example C1-C2-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy.

Alkylthio means saturated, straight-chain or branched alkylthio radicals having 1 to 8 carbon atoms, for example C1-C6-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

haloalkylthio means straight-chain or branched alkylthio groups having 1 to 8 carbon atoms (as mentioned above), where in these groups some or all of the hydrogen atoms may be replaced by halogen atoms as mentioned above, for example C₁-C₂-haloalkylthio such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio.

Heteroaryl means 2-furyl, 3-furyl, 2-thienyl, 3-thienyl, 1-pyrrolyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 5-imidazolyl, 1,2,4-oxadiazol-3-yl, 1,2,4-oxadiazol-5-yl, 1,2,4-thiadiazol-3-yl, 1,2,4-thiadiazol-5-yl, 1,3,4-oxadiazol-2-yl, 1,3,4-thiadiazol-2-yl, 1,2,4-triazol-1-yl, 1,2,4-triazol-3-yl, 1,2,4-triazol-4-yl, 1,2,4-triazol-5-yl, 1,2,3-triazol-1-yl, 1,2,3-triazol-2-yl, 1,2,3-triazol-4-yl, tetrazol-1-yl, tetrazol-2-yl, tetrazol-5-yl, indol-1-yl, indol-2-yl, indol-3-yl, isoindol-1-yl, isoindol-2-yl, benzofur-2-yl, benzothiophen-2-yl, benzofur-3-yl, benzothiophen-3-yl, benzoxazol-2-yl, benzothiazol-2-yl, benzimidazol-2-yl, indazol-1-yl, indazol-2-yl, indazol-3-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl or 1,2,4-triazin-6-yl. This heteroaryl is in each case unsubstituted or in each case mono- or polysubstituted by identical or different radicals selected from the group consisting of fluorine, chlorine, bromine, iodine, cyano, hydroxyl, mercapto, amino, methyl, ethyl, n-propyl, isopropyl, n-butyl, isobutyl, sec-butyl, tert-butyl, cyclopropyl, 1-chlorocyclopropyl, vinyl, ethynyl, methoxy, ethoxy, isopropoxy, methylthio, ethylthio, trifluoromethylthio, chlorodifluoromethyl, dichlorofluoromethyl, chlorofluoromethyl, chloromethyl, dichloromethyl, trichloromethyl, fluoromethyl, difluoromethyl, trifluoromethyl, 2,2,2-trifluoroethyl, trifluoromethoxy, trifluoromethylthio, 2,2,2-trifluoroethoxy, 2,2-dichloro-2-fluoroethyl, 2,2-difluoro-2-chloroethyl, 2-chloro-2-fluoroethyl, 2,2,2-trichloroethyl, 2,2,2-trifluoroethyl, 2-fluoroethyl, 2,2-difluoroethyl, 2-methoxyethoxy, acetyl, propionyl, methoxycarbonyl, ethoxycarbonyl, N-methylamino, N,N-dimethylamino, N-ethylamino, N,N-diethylamino, aminocarbonyl, methylaminocarbonyl, dimethylaminocarbonyl, dimethylcarbamoylamino, methoxycarbonylamino, methoxycarbonyloxy, ethoxycarbonylamino, ethoxycarbonyloxy, methylsulfamoyl, dimethylsulfamoyl, phenyl or phenoxy.

Depending inter alia on the nature of the substituents, the compounds of the formula (I) can be present as geometrical and/or optical isomers or isomer mixtures of varying composition which, if desired, can be separated in a customary manner. The present invention provides both the pure isomers and the isomer mixtures, their preparation and use and compositions comprising them. For the sake of simplicity, however, compounds of the formula (I) are always referred to below, although both the pure compounds and also, if appropriate, mixtures having different proportions of isomeric compounds are meant.

Where a group is substituted by a plurality of radicals, this means that this group is substituted by one or more identical or different representatives of the radicals mentioned.

Preference is given to compounds of the formulae (I-a), (I-b), (I-c), (I-d), (I-e), (I-f) and (I-g):

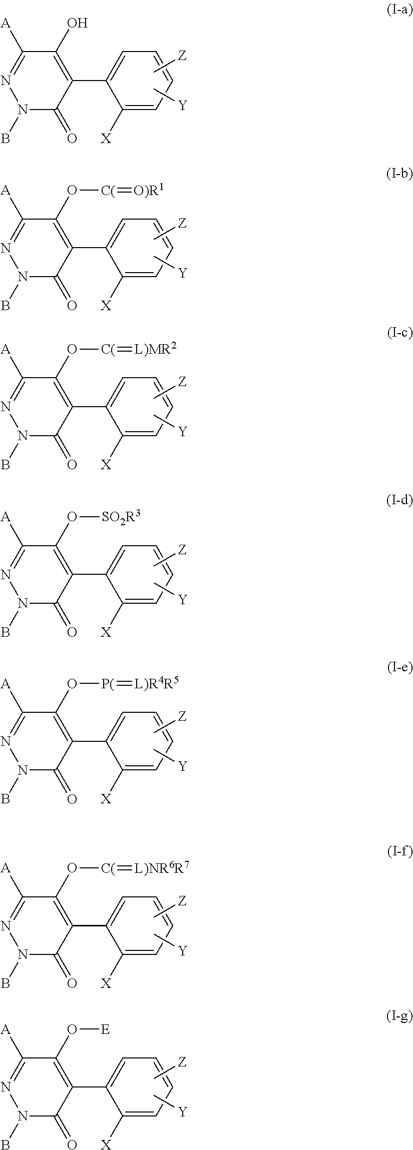

Preference is also given to compounds of the formula (I) in which
A is hydrogen or (C₁-C₆)-alkyl;
B is hydrogen or (C₁-C₆)-alkyl;
n is 0, 1, 2 or 3;
G is hydrogen, C(═O)R¹, C(=L)MR², SO₂R³, P(=L)R⁴R⁵, C(=L)NR⁶R⁷ or E;
E is Na⁺, K⁺, (Mg²⁺)_{1/2}, (Ca²⁺)_{1/2}, R¹³R¹⁴R¹⁵R¹⁶N⁺ or NH₄⁺;
R¹³, R¹⁴, R¹⁵ and R¹⁶ independently of one another are (C₁-C₆)-alkyl or benzyl;
L is oxygen;
M is oxygen;
R¹ is (C₁-C₆)-alkyl substituted by n halogen atoms or (C₃-C₆)-cycloalkyl, phenyl or phenyl-(C₁-C₄)-alkyl, each of which is substituted by n radicals from the group consisting of halogen, (C₁-C₄)-alkyl and (C₁-C₄)-alkoxy;
R² is (C₁-C₆)-alkyl substituted by n halogen atoms or (C₃-C₆)-cycloalkyl, phenyl or benzyl, each of which is substituted by n radicals from the group consisting of halogen, (C₁-C₄)-alkyl and (C₁-C₄)-alkoxy;
R³, R⁴ and R⁵ are each independently of one another (C₁-C₆)-alkyl substituted by n halogen atoms or benzyl or phenyl substituted by n radicals from the group consisting of halogen, (C₁-C₄)-alkyl and (C₁-C₄)-alkoxy;
R⁶ and R⁷ are each independently of one another hydrogen, (C₁-C₆)-alkyl substituted by n halogen atoms or benzyl or phenyl substituted by n radicals from the group consisting of halogen, (C₁-C₄)-alkyl and (C₁-C₄)-alkoxy;
X is methyl, ethyl or methoxy;
Y is halogen;
Z is halogen.

Particular preference is given to compounds of the formula (I) in which
A is hydrogen, methyl, ethyl;
B is methyl or ethyl;
n is 0, 1, 2 or 3;
G is hydrogen, C(═O)R¹, C(=L)MR², SO₂R³, P(=L)R⁴R⁵, C(=L)NR⁶R⁷ or E;
E is Na⁺, K⁺, (Mg²⁺)_{1/2}, (Ca²⁺)_{1/2}, (CH₃)₄N⁺ or NH₄⁺;
L is oxygen;
M is oxygen;
R¹ is (C₁-C₆)-alkyl or (C₃-C₆)-cycloalkyl;
R² is (C₁-C₆)-alkyl or (C₃-C₆)-cycloalkyl;
R³, R⁴ and R⁵ are each independently of one another (C₁-C₆)-alkyl, phenyl or benzyl;
R⁶ and R⁷ are each independently of one another hydrogen, (C₁-C₆)-alkyl, phenyl or benzyl;
X is methyl or ethyl;
Y is fluorine, chlorine, bromine or iodine,
Z is fluorine, bromine or iodine.

Very particular preference is given to the compounds of the formula (I) listed in Tables 1 to 30, which can be obtained analogously to the methods described herein.

The abbreviations used denote:
Bz=benzyl c-Pr=cyclopropyl Et=ethyl
i-Bu=isobutyl t-Bu=tertiary butyl i-Pr=isopropyl
Me=methyl Ph=phenyl

TABLE 1
Compounds of the formula (I) according to the invention
in which G is hydrogen and A is hydrogen and B is methyl.

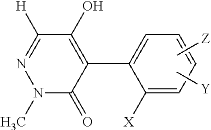

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 2
Compounds of the formula (I) according to the invention
in which G is hydrogen, A is hydrogen and B is ethyl.

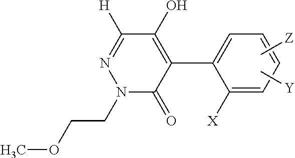

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 3
Compounds of the formula (I) according to the invention
in which G is hydrogen, A is hydrogen and B is n-propyl.

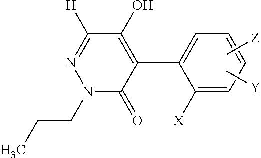

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 4
Compounds of the formula (I) according to the invention in
which G is hydrogen, A is hydrogen and B is i-propyl.

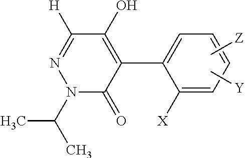

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 5
Compounds of the formula (I) according to the invention in
which G is hydrogen, A is hydrogen and B is 2-methoxyethyl.

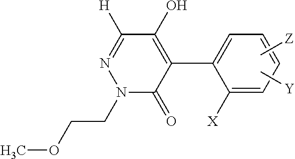

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 6
Compounds of the formula (I) according to the invention in which G
is hydrogen, A is hydrogen and B is 2-ethoxyethyl.

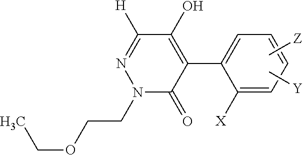

| No | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 7
Compounds of the formula (I) according to the invention in which G
is hydrogen and A is methyl and B is methyl.

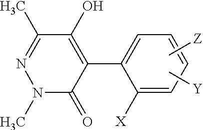

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 8
Compounds of the formula (I) according to the invention in which G
is hydrogen, A is methyl and B is ethyl.

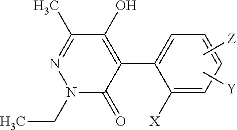

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 9
Compounds of the formula (I) according to the invention in which G
is hydrogen, A is methyl and B is n-propyl.

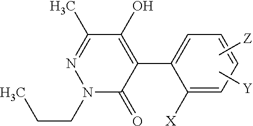

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 10
Compounds of the formula (I) according to the invention in which G
is hydrogen, A is methyl and B is i-propyl.

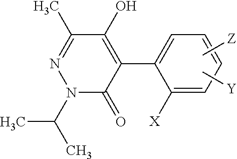

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 11
Compounds of the formula (I) according to the invention in which G
is hydrogen, A is methyl and B is 2-methoxyethyl.

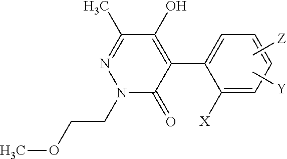

| No . | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 12
Compounds of the formula (I) according to the invention in which G
is hydrogen, A is methyl and B is 2-ethoxyethyl.

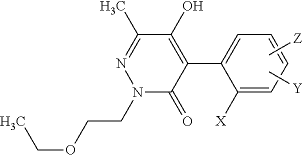

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 13
Compounds of the formula (I) according to the invention in which G
is hydrogen and A is ethyl and B is methyl.

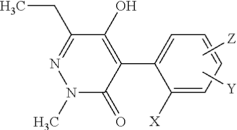

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 14
Compounds of the formula (I) according to the invention in which G
is hydrogen, A is ethyl and B is ethyl.

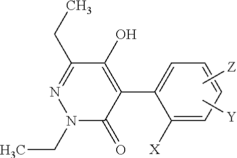

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 15
Compounds of the formula (I) according to the invention in which G
is hydrogen, A is ethyl and B is n-propyl.

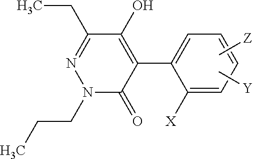

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 16
Compounds of the formula (I) according to the invention in which G
is hydrogen, A is ethyl and B is i-propyl.

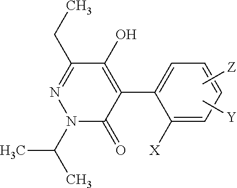

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 17
Compounds of the formula (I) according to the invention in which G
is hydrogen, A is ethyl and B is 2-methoxyethyl.

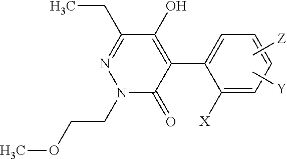

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 18
Compounds of the formula (I) according to the invention in which G
is hydrogen, A is ethyl and B is 2-ethoxyethyl.

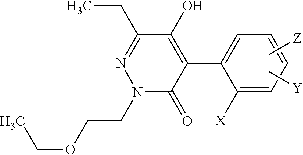

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 19
Compounds of the formula (I) according to the invention in which G
is hydrogen and A is n-propyl and B is methyl.

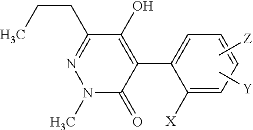

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 20
Compounds of the formula (I) according to the invention in which G
is hydrogen, A is n-propyl and B is ethyl.

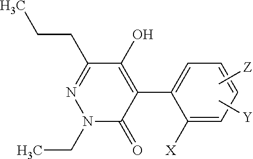

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 21
Compounds of the formula (I) according to the invention in which G
is hydrogen, A is n-propyl and B is n-propyl.

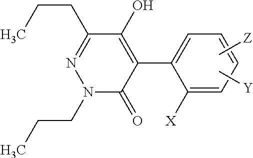

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 22
Compounds of the formula (I) according to the invention in which G
is hydrogen, A is n-propyl and B is i-propyl.

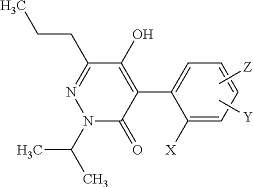

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 23
Compounds of the formula (I) according to the invention in which G
is hydrogen, A is n-propyl and B is 2-methoxyethyl.

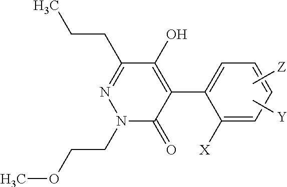

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 24
Compounds of the formula (I) according to the invention in which G
is hydrogen, A is n-propyl and B is 2-ethoxyethyl.

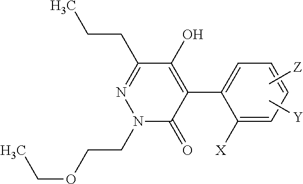

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 25
Compounds of the formula (I) according to the invention in which G
is hydrogen and A is i-propyl and B is methyl.

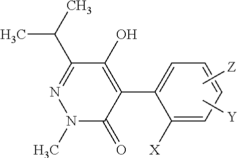

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 26
Compounds of the formula (I) according to the invention in which G
is hydrogen, A is i-propyl and B is ethyl.

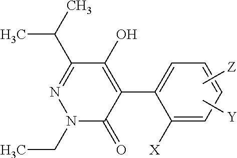

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 27
Compounds of the formula (I) according to the invention in which G
is hydrogen, A is i-propyl and B is n-propyl.

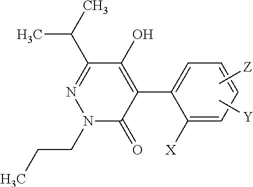

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 28
Compounds of the formula (I) according to the invention in which G
is hydrogen, A is i-propyl and B is i-propyl.

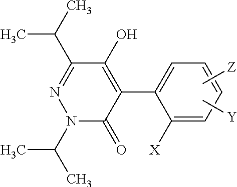

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 29
Compounds of the formula (I) according to the invention in which G
is hydrogen, A is i-propyl and B is 2-methoxyethyl.

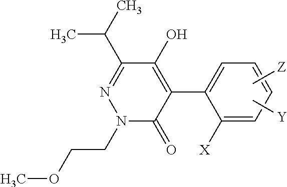

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

TABLE 30
Compounds of the formula (I) according to the invention in which G
is hydrogen, A is i-propyl and B is 2-ethoxyethyl.

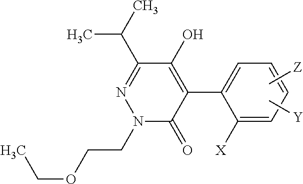

| No. | X | Y | Z |
|---|---|---|---|
| 1 | Me | 4-Cl | 6-Br |
| 2 | Me | 4-Cl | 6-I |
| 3 | Me | 4-Br | 6-Br |
| 4 | Me | 4-I | 6-Cl |
| 5 | Me | 4-Br | 6-Cl |
| 6 | Me | 4-Br | 5-F |
| 7 | Me | 4-Cl | 5-Br |
| 8 | Me | 4-F | 6-Br |
| 9 | Me | 4-Br | 5-Cl |
| 10 | Me | 4-F | 5-F |
| 11 | Me | 3-F | 4-F |
| 12 | Me | 4-F | 5-Br |
| 13 | Et | 4-Cl | 6-Br |
| 14 | Et | 4-Cl | 6-I |
| 15 | Et | 4-Br | 6-Br |
| 16 | Et | 4-I | 6-Cl |
| 17 | Et | 4-Br | 6-Cl |
| 18 | OMe | 5-F | 6-F |
| 19 | OMe | 3-Cl | 5-F |
| 20 | OMe | 3-F | 5-Cl |
| 21 | OEt | 3-F | 5-F |
| 22 | OMe | 3-F | 5-F |

Very particular preference is also given to compounds of Tables 1 to 30 above in which G is C(═O)R¹, C(=L)LR², SO₂R³, P(=L)R⁴R⁵, C(=L)NR⁶R⁷ or E.

Collections of compounds of the formula (I) and/or salts thereof which can be synthesized by the aforementioned reactions can also be prepared in a parallel manner, it being possible for this to take place in a manual, partly automated or completely automated manner. In this connection, it is, for example, possible to automate the reaction procedure, the work-up or the purification of the products and/or intermediates. Overall, this is understood as meaning a procedure as described, for example, by D. Tiebes in Combinatorial Chemistry—Synthesis, Analysis, Screening (editor Günther Jung), Verlag Wiley 1999, on pages 1 to 34.

For the parallel reaction procedure and work-up, it is possible to use a series of commercially available instruments, for example Calpyso reaction blocks from Barnstead International, Dubuque, Iowa 52004-0797, USA or reaction stations from Radleys, Shirehill, Saffron Walden, Essex, CB 11 3AZ, England or MultiPROBE Automated Workstations from Perkin Elmer, Waltham, Mass. 02451, USA. For the parallel purification of compounds of the formula (I) and salts thereof or of intermediates produced during the preparation, there are available, inter alia, chromatography apparatuses, for example from ISCO, Inc., 4700 Superior Street, Lincoln, Neb. 68504, USA.

The apparatuses listed lead to a modular procedure in which the individual process steps are automated, but between the process steps manual operations have to be carried out. This can be circumvented by using partly or completely integrated automation systems in which the respective automation modules are operated, for example, by robots. Automation systems of this type can be acquired, for example, from Caliper, Hopkinton, Mass. 01748, USA.

The implementation of single or several synthesis steps can be supported through the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Besides the methods described here, the preparation of compounds of the formula (I) and salts thereof can take place completely or partially by solid-phase supported methods. For this purpose, individual intermediates or all intermediates in the synthesis or a synthesis adapted for the corresponding procedure are bonded to a synthesis resin. Solid-phase supported synthesis methods are sufficiently described in the specialist literature, e.g. Barry A. Bunin in “The Combinatorial Index”, Verlag Academic Press, 1998 and Combinatorial Chemistry—Synthesis, Analysis, Screening (editor Günther Jung), Verlag Wiley, 1999. The use of solid-phase supported synthesis methods permits a series of protocols known in the literature, which again can be carried out manually or in an automated manner. The reactions can be carried out, for example, by means of IRORI technology in microreactors from Nexus Biosystems, 12140 Community Road, Poway, Calif. 92064, USA.

Both on a solid phase and in liquid phase can the procedure of individual or several synthesis steps be supported through the use of microwave technology. The specialist literature describes a series of experimental protocols, for example in Microwaves in Organic and Medicinal Chemistry (editor C. O. Kappe and A. Stadler), Verlag Wiley, 2005.

The preparation according to the process described here produces compounds of the formula (I) and their salts in the form of substance collections which are called libraries. The present invention also provides libraries which comprise at least two compounds of the formula (I) and their salts.

The compounds of the formula (I) according to the invention (and/or their salts), hereinbelow also referred to together as “compounds according to the invention”, have excellent herbicidal efficacy against a broad spectrum of economically important monocotyledonous and dicotyledonous annual harmful plants. The active compounds act efficiently even on perennial harmful plants which produce shoots from rhizomes, root stocks and other perennial organs and which are difficult to control.

The present invention therefore also relates to a method for controlling unwanted plants or for regulating the growth of plants, preferably in crops of plants, where one or more compound(s) according to the invention is/are applied to the plants (for example harmful plants such as monocotyledonous or dicotyledonous weeds or undesired crop plants), to the seeds (for example grains, seeds or vegetative propagules such as tubers or shoot parts with buds) or to the area on which the plants grow (for example the area under cultivation). In this context, the compounds according to the invention can be applied for example pre-sowing (if appropriate also by incorporation into the soil), pre-emergence or post-emergence. Specific examples may be mentioned of some representatives of the monocotyledonous and dicotyledonous weed flora which can be controlled by the compounds according to the invention, without the enumeration being restricted to certain species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

If the compounds according to the invention are applied to the soil surface before germination, the weed seedlings are either prevented completely from emerging or else the weeds grow until they have reached the cotyledon stage, but then their growth stops, and, eventually, after three to four weeks have elapsed, they die completely.

If the active compounds are applied post-emergence to the green parts of the plants, growth stops after the treatment, and the harmful plants remain at the growth stage of the point of time of application, or they die completely after a certain time, so that in this manner competition by the weeds, which is harmful to the crop plants, is eliminated very early and in a sustained manner.

Although the compounds according to the invention display an outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, in particular Zea and Triticum, are damaged only to an insignificant extent, or not at all, depending on the structure of the respective compound according to the invention and its application rate. This is why the present compounds are highly suitable for the selective control of unwanted plant growth in plant crops such as agriculturally useful plants or ornamentals.

Moreover, the compounds according to the invention (depending on their respective structure and the application rate applied) have outstanding growth-regulatory properties in crop plants. They engage in the plant's metabolism in a regulatory fashion and can therefore be employed for the influencing, in a targeted manner, of plant constituents and for facilitating harvesting, such as, for example, by triggering desiccation and stunted growth. Moreover, they are also suitable for generally controlling and inhibiting unwanted vegetative growth without destroying the plants in the process. Inhibiting the vegetative growth plays an important role in many monocotyledonous and dicotyledonous crops since for example lodging can be reduced, or prevented completely, hereby.

By virtue of their herbicidal and plant-growth-regulatory properties, the active compounds can also be employed for controlling harmful plants in crops of genetically modified plants or plants modified by conventional mutagenesis. In general, the transgenic plants are distinguished by especially advantageous properties, for example by resistances to certain pesticides, mainly certain herbicides, resistances to plant diseases or causative organisms of plant diseases, such as certain insects or microorganisms such as fungi, bacteria or viruses. Other specific characteristics relate, for example, to the harvested material with regard to quantity, quality, storability, composition and specific constituents. Thus, transgenic plants are known whose starch content is increased, or whose starch quality is altered, or those where the harvested material has a different fatty acid composition.

It is preferred to use the compounds according to the invention or their salts in economically important transgenic crops of useful plants and ornamentals, for example of cereals such as wheat, barley, rye, oats, millet, rice, cassava and corn or else crops of sugar beet, cotton, soybean, oilseed rape, potato, tomato, peas and other vegetables. It is preferred to employ the compounds according to the invention as herbicides in crops of useful plants which are resistant, or have been made resistant by recombinant means, to the phytotoxic effects of the herbicides.

Conventional methods of generating novel plants which have modified properties in comparison to plants occurring to date consist, for example, in traditional breeding methods and the generation of mutants. Alternatively, novel plants with altered properties can be generated with the aid of recombinant methods (see, for example, EP-A-0221044, EP-A-0131624). For example, the following have been described in several cases:
the modification, by recombinant technology, of crop plants with the aim of modifying the starch synthesized in the plants (for example WO 92/11376, WO 92/14827, WO 91/19806),
transgenic crop plants which are resistant to certain herbicides of the glufosinate type (cf., for example, EP-A-0242236, EP-A-242246) or of the glyphosate type (WO 92/00377) or of the sulfonylurea type (EP-A-0257993, U.S. Pat. No. 5,013,659),
transgenic crop plants, for example cotton, with the capability of producing Bacillus thuringiensis toxins (Bt toxins), which make the plants resistant to certain pests (EP-A-0142924, EP-A-0193259),
transgenic crop plants with a modified fatty acid composition (WO 91/13972),
genetically modified crop plants with novel constituents or secondary metabolites, for example novel phytoalexins, which bring about an increased disease resistance (EPA 309862, EPA0464461),
genetically modified plants with reduced photorespiration which feature higher yields and higher stress tolerance (EPA 0305398),
transgenic crop plants which produce pharmaceutically or diagnostically important proteins (“molecular pharming”),
transgenic crop plants which are distinguished by higher yields or better quality,
transgenic crop plants which are distinguished by a combination, for example of the abovementioned novel properties (“gene stacking”).

A large number of molecular-biological techniques by means of which novel transgenic plants with modified properties can be generated are known in principle; see, for example, I. Potrykus and G. Spangenberg (eds.) Gene Transfer to Plants, Springer Lab Manual (1995), Springer Verlag Berlin, Heidelberg. or Christou, “Trends in Plant Science” 1 (1996) 423-431.

To carry out such recombinant manipulations, nucleic acid molecules which allow mutagenesis or a sequence change by recombination of DNA sequences can be introduced into plasmids. For example, base substitutions can be carried out, part-sequences can be removed, or natural or synthetic sequences may be added with the aid of standard methods. To link the DNA fragments with one another, it is possible to add adapters or linkers to the fragments; see, for example, Sambrook et al., 1989, Molecular Cloning, A Laboratory Manual, 2nd ed., Cold Spring Harbor Laboratory Press, Cold Spring Harbor, N.Y.; or Winnacker “Gene und Klone”, VCH Weinheim 2nd ed., 1996.

For example, the generation of plant cells with a reduced activity of a gene product can be achieved by expressing at least one corresponding antisense RNA, a sense RNA for achieving a cosuppression effect or by expressing at least one suitably constructed ribozyme which specifically cleaves transcripts of the abovementioned gene product. To this end, it is possible to use DNA molecules which encompass the entire coding sequence of a gene product inclusive of any flanking sequences which may be present, and also DNA molecules which only encompass portions of the coding sequence, it being necessary for these portions to be long enough to have an antisense effect in the cells. The use of DNA sequences which have a high degree of homology to the coding sequences of a gene product, but are not completely identical to them, is also possible.

When expressing nucleic acid molecules in plants, the protein synthesized can be localized in any desired compartment of the plant cell. However, to achieve localization in a particular compartment, it is possible, for example, to link the coding region with DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants can be plants of any desired plant species, i.e. not only monocotyledonous, but also dicotyledonous, plants.

Thus, transgenic plants can be obtained whose properties are altered by overexpression, suppression or inhibition of homologous (=natural) genes or gene sequences or the expression of heterologous (=foreign) genes or gene sequences.

It is preferred to employ the compounds according to the invention in transgenic crops which are resistant to growth regulators such as, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active compounds.

When the active compounds according to the invention are used in transgenic crops, effects are frequently observed—in addition to the effects on harmful plants which can be observed in other crops—which are specific for the application in the transgenic crop in question, for example a modified or specifically widened spectrum of weeds which can be controlled, modified application rates which may be employed for application, preferably good combinability with the herbicides to which the transgenic crop is resistant, and an effect on growth and yield of the transgenic crop plants.

The invention therefore also relates to the use of the compounds according to the invention as herbicides for controlling harmful plants in transgenic crop plants.

The compounds according to the invention can be used in the form of wettable powders, emulsifiable concentrates, sprayable solutions, dusting products or granules in the customary formulations. The invention therefore also provides herbicidal and plant-growth-regulating compositions which comprise the compounds according to the invention.

The compounds according to the invention can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW) such as oil-in-water and water-in-oil emulsions, sprayable solutions, suspension concentrates (SC), oil- or water-based dispersions, oil-miscible solutions, capsule suspensions (CS), dusting products (DP), seed-dressing products, granules for scattering and soil application, granules (GR) in the form of microgranules, spray granules, coated granules and adsorption granules, water-dispersible granules (WG), water-soluble granules (SG), ULV formulations, microcapsules and waxes.

These individual types of formulation are known in principle and are described, for example, in: Winnacker-Küchler, “Chemische Technologie” [Chemical technology], Volume 7, C. Hanser Verlag Munich, 4th Ed. 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The necessary formulation assistants, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H.v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Interface-active ethylene oxide adducts], Wiss. Verlagsgesell., Stuttgart 1976; Winnacker-Küchler, “Chemische Technologie”, Volume 7, C. Hanser Verlag Munich, 4th Ed. 1986.

Based on these formulations, it is also possible to produce combinations with other pesticidally active compounds, such as, for example, insecticides, acaricides, herbicides, fungicides, and also with safeners, fertilizers and/or growth regulators, for example in the form of a finished formulation or as a tank mix. Suitable safeners are, for example, mefenpyr-diethyl, cyprosulfamide, isoxadifen-ethyl, cloquintocet-mexyl and dichlormid.

Wettable powders are preparations which can be dispersed uniformly in water and, as well as the active compound, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurinate. To prepare the wettable powders, the herbicidally active compounds are ground finely, for example in customary apparatuses such as hammer mills, blower mills and air-jet mills, and simultaneously or subsequently mixed with the formulation assistants.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents with addition of one or more surfactants of the ionic and/or nonionic type (emulsifiers). The emulsifiers used may, for example, be: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusts are obtained by grinding the active compound with finely distributed solid substances, for example talc, natural clays, such as kaolin, bentonite and pyrophyllite, or diatomaceous earth.

Suspension concentrates may be water- or oil-based. They may be prepared, for example, by wet grinding by means of commercial bead mills and optional addition of surfactants as have, for example, already been listed above for the other formulation types.

Emulsions, for example oil-in-water emulsions (EW), can be prepared, for example, by means of stirrers, colloid mills and/or static mixers using aqueous organic solvents and optionally surfactants, as have, for example, already been listed above for the other formulation types.

Granules can be prepared either by spraying the active compound onto granulated inert material capable of adsorption or by applying active compound concentrates to the surface of carrier substances, such as sand, kaolinites or granulated inert material, by means of adhesives, for example polyvinyl alcohol, sodium polyacrylate or mineral oils. Suitable active compounds can also be granulated in the manner customary for the preparation of fertilizer granules—if desired as a mixture with fertilizers.

Water-dispersible granules are prepared generally by the customary processes such as spray-drying, fluidized bed granulation, pan granulation, mixing with high-speed mixers and extrusion without solid inert material.

For the preparation of pan, fluidized bed, extruder and spray granules, see, for example, processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th Ed., McGraw-Hill, New York 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical formulations contain generally from 0.1 to 99% by weight, in particular from 0.1 to 95% by weight, of compounds according to the invention.

In wettable powders, the active compound concentration is, for example, from about 10 to 90% by weight, the remainder to 100% by weight consisting of customary formulation components. In the case of emulsifiable concentrates, the active compound concentration can be from about 1 to 90, preferably from 5 to 80, % by weight. Formulations in the form of dusts comprise from 1 to 30% by weight of active compound, preferably usually from 5 to 20% by weight of active compound; sprayable solutions contain from about 0.05 to 80% by weight, preferably from 2 to 50% by weight of active compound. In the case of water-dispersible granules, the active compound content depends partially on whether the active compound is present in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary adhesives, wetting agents, dispersants, emulsifiers, penetrants, preservatives, antifreeze agents and solvents, fillers, carriers and dyes, defoamers, evaporation inhibitors and agents which influence the pH and the viscosity.

Based on these formulations, it is also possible to produce combinations with other pesticidally active compounds, such as, for example, insecticides, acaricides, herbicides, fungicides, and also with safeners, fertilizers and/or growth regulators, for example in the form of a finished formulation or as a tank mix.

Active compounds which can be employed in combination with the compounds according to the invention in mixed formulations or in the tank mix are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoen desaturase, photosystem I, photosystem II, protoporphyrinogen oxidase, as are described in, for example, Weed Research 26 (1986) 441-445 or “The Pesticide Manual”, 14th edition, The British Crop Protection Council and the Royal Soc. of Chemistry, 2003 and the literature cited therein. Known herbicides or plant growth regulators which can be combined with the compounds according to the invention are, for example, the following active compounds (the compounds are either designated by the common name according to the International Organization for Standardization (ISO) or by the chemical name, or by the code number) and always comprise all use forms such as acids, salts, esters and isomers such as stereoisomers and optical isomers. Here, by way of example, one and in some cases a plurality of use forms are mentioned:

acetochlor, acibenzolar, acibenzolar-S-methyl, acifluorfen, acifluorfen-sodium, aclonifen, alachlor, allidochlor, alloxydim, alloxydim-sodium, ametryn, amicarbazone, amidochlor, amidosulfuron, aminocyclopyrachlor, aminopyralid, amitrole, ammonium sulfamate, ancymidol, anilofos, asulam, atrazine, azafenidin, azimsulfuron, aziprotryn, BAH-043, BAS-140H, BAS-693H, BAS-714H, BAS-762H, BAS-776H, BAS-800H, beflubutamid, benazolin, benazolin-ethyl, bencarbazone, benfluralin, benfuresate, bensulide, bensulfuron-methyl, bentazone, benzfendizone, benzobicyclon, benzofenap, benzofluor, benzoylprop, bicyclopyrone, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat chloride, chlornitrofen, chlorophthalim, chlorthal-dimethyl, chlorotoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymron, dalapon, daminozide, dazomet, n-decanol, desmedipham, desmetryn, detosyl-pyrazolate (DTP), diallate, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, dipropetryn, diquat, diquat dibromide, dithiopyr, diuron, DNOC, eglinazine-ethyl, endothal, EPTC, esprocarb, ethalfluralin, ethametsulfuron-methyl, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr, fluroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, L-glufosinate, L-glufosinate-ammonium, glufosinate-ammonium, glyphosate, glyphosate-isopropylammonium, H-9201, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HNPC-9908, HOK-201, HW-02, imazamethabenz, imazamethabenz-methyl, imazamox, imazapic, imazapyr, imazaquin, imazethapyr, imazosulfuron, inabenfide, indanofan, indaziflam, indoleacetic acid (IAA), 4-indol-3-ylbutyric acid (IBA), iodosulfuron, iodosulfuron-methyl-sodium, ioxynil, ipfencarbazone, isocarbamid, isopropalin, isoproturon, isouron, isoxaben, isoxachlortole, isoxaflutole, isoxapyrifop, KUH-043, KUH-071, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat chloride, mesosulfuron, mesosulfuron-methyl, mesotrione, methabenzthiazuron, metam, metamifop, metamitron, metazachlor, metazosulfuron, methazole, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monuron, MT 128, MT-5950, i.e. N-[3-chloro-4-(1-methylethyl)phenyI]-2-methylpentanamide, NGGC-011, naproanilide, napropamide, naptalam, NC-310, i.e. 4-(2,4-dichlorobenzoyl)-1-methyl-5-benzyloxypyrazole, NC-620, neburon, nicosulfuron, nipyraclofen, nitralin, nitrofen, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorten, paclobutrazol, paraquat, paraquat dichloride, pelargonic acid (nonanoic acid), pendimethalin, pendralin, penoxsulam, pentanochlor, pentoxazone, pertluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazol, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron-ethyl, pyrazoxyfen, pyribambenz, pyribambenz-isopropyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, sulcotrione, sulfallate (CDEC), sulfentrazone, sulfometuron, sulfometuron-methyl, sulfosate (glyphosate-trimesium), sulfosulfuron, SYN-523, SYP-249, SYP-298, SYP-300, tebutam, tebuthiuron, tecnazene, tefuryltrione, tembotrione, tepraloxydim, terbacil, terbucarb, terbuchlor, terbumeton, terbuthylazine, terbutryn, TH-547, i.e. propynsulfuron, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triallate, triasulfuron, triaziflam, triazofenamide, tribenuron, tribenuron-methyl, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0166, ZJ-0270, ZJ-0543, ZJ-0862 and the following compounds

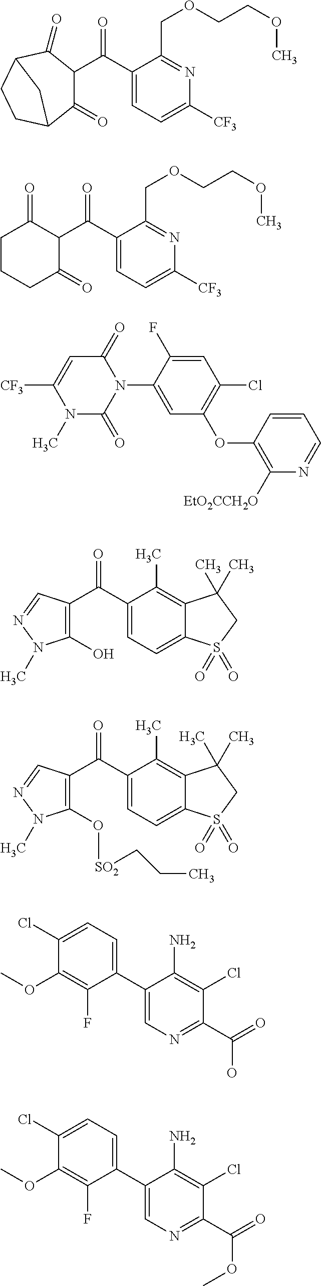

For application, the formulations present in commercial form are, if appropriate, diluted in a customary manner, for example in the case of wettable powders, emulsifiable concentrates, dispersions and water-dispersible granules with water. Preparations in the form of dusts, granules for soil application or granules for broadcasting and sprayable solutions are usually not diluted further with other inert substances prior to application.

The required application rate of the compounds of the formula (I) varies according to the external conditions such as, inter alia, temperature, humidity and the type of herbicide used. It may vary within wide limits, for example between 0.001 and 1.0 kg/ha or more of active substance; however, preferably it is between 0.005 and 750 g/ha.

In addition to the herbicidal action, the compounds according to the invention also have good insecticidal activity. Accordingly, the present invention furthermore provides insecticidal compositions comprising the compounds according to the invention and their use as insecticides.

The examples below illustrate the invention:

### CHEMICAL EXAMPLES

### Preparation of 1-(2-Br-4-chlorophenylacetic acid) 1-methylhydrazide

2.0. g (7.59 mmol) of (2-bromo-4-chloro-6-methylphenylacetic acid were initially charged in 50 ml of CH2Cl2, and 2 drops of DMF were added. 1.3. eq of oxalyl chloride were then added, and the mixture was heated at the boil under reflux until the evolution of gas had ceased. The solvent was then removed under reduced pressure and the residue was taken up in 10 ml of CH2Cl2 (solution A). 0.35 g (7.59 mmol) of methylhydrazine were initially charged in 50 ml of CH2Cl2, and 1 eq of triethylamine and 5 mol % of DMAP were added. At 0° C., solution A was added dropwise, and the mixture was then stirred under reflux for 1 h. A solution of 6 g of ammonium chloride in 50 ml of water was then added and the organic phase was separated off, giving 2.19 g (yield 99% of theory) of a pale yellow solid:

1 H-NMR (400 MHz, DMSO-d6): 7.55 (d, 1H), 7.33 (d, 1H), 4.89 (s, 2H), 4.07 (s, 2H), 3.03 (s, 3H), 2.22 (s, 3H).

2.19 g (7.5 mmol) of 1-(2-bromo-4-chlorophenylacetic acid) 1-methylhydrazide were dissolved in 20 ml of anhydrous ethanol, and 1 eq of methyl pyruvate was added. The mixture was then heated to the boil under reflux for 2 h, and the reaction solution was allowed to stand overnight. A precipitate was formed. This precipitate was filtered off. This gave 1.426 g (yield 50.6% of theory). The mother liquor obtained was then purified chromatographically (silica gel, gradient EtOAc/n-heptane), giving another 1.672 g (purity 80%) of the desired target product.

¹H-NMR (400 MHz, DMSO-d₆): 7.57 (d, 1H), 7.35 (d, 1H), 4.08 (s, 2H), 3.79 (s, 3H), 3.36 (s, 3H) 3.03 (s, 3H), 2.28 (s, 3H), 2.24 (s, 3H).

At 0° C., a solution of 2.82 g (7.5 mmol) of methyl 2-{[2-(2-bromo-4-chloro-6-ethylphenyl)acetyl]methylhydrazono}propionate in 10 ml of DMF was added dropwise to a solution of 2.0 eq of potassium t-butoxide in 10 ml of DMF over the course of 2 h. The mixture was then warmed to room temperature and stirred for 30 min. The reaction solution was then added to a mixture of 50 g of ice-water and 50 ml of 1N hydrochloric acid and then extracted twice with in each case 250 ml of ethyl acetate. The combined organic phases were washed with 50 ml of saturated sodium chloride solution and then concentrated under reduced pressure. The brown solid formed was suspended in n-heptane and then filtered off. This gave 1.65 g (yield 64.1% of theory) of the compound I-1-a-1 according to the invention.

¹H-NMR (400 MHz, DMSO-d₆): 7.67 (d, 1H), 7.44 (d, 1H), 3.59 (s, 3H), 2.23 (s, 3H), 2.04 (s, 3H).

The following compounds were prepared analogously to the examples given above:

TABLE 31
Compounds of the formula (I) according to the invention in
which G is hydrogen.
(I-a)

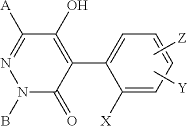

| No. | X | Y | Z | A | B | Analytical data |
|---|---|---|---|---|---|---|
| I-1-a-1 | Me | 6-Br | 4-Cl | Me | Me | ¹H-NMR (400 MHz, DMSO-d₆): |
| | | | | | | 7.67 (d, 1H), 7.44 (d, 1H), 3.59 |
| | | | | | | (s, 3H), 2.23 (s, 3H), 2.04 (s, 3H) |
| I-1-a-2 | Et | 6-Cl | 4-Br | Me | Me | ¹H-NMR (400 MHz, DMSO-d₆): |
| | | | | | | 7.62 (d, 1H), 7.50 (d, 1H), 3.58 |
| | | | | | | (s, 3H), 2.33 (m, 2H), 2.21 (s, |
| | | | | | | 3H), 1.03 (dt, 3H) |
| I-1-a-3 | Et | 6-Br | 4-Cl | Me | Me | ¹H-NMR (400 MHz, DMSO-d₆): |
| | | | | | | 7.65 (d, 1H), 7.40 (d, 1H), 3.56 |
| | | | | | | (s, 3H), 2.33 (m, 2H), 2.23 (s, |
| | | | | | | 3H), 1.02 (dt, 3H) |
| I-1-a-4 | Me | 6-Cl | 4-Br | Me | Me | ¹H-NMR (400 MHz, DMSO-d₆): |
| | | | | | | 7.49 (d, 1H), 7.38 (d, 1H), 3.72 |
| | | | | | | (s, 3H), 2.21 (s, 3H), 2.18 (t, 3H) |
| I-1-a-5 | Et | 6-Cl | 4-I | Me | Me | ¹H-NMR (400 MHz, DMSO-d₆): |
| | | | | | | 7.74 (d, 1H), 7.62 (d, 1H), 3.56 |
| | | | | | | (s, 3H), 2.30 (m, 2H), 2.23 (s, |
| | | | | | | 3H), 1.00 (dt, 3H) |
| I-1-a-6 | Et | 6-I | 4-Cl | Me | Me | ¹H-NMR (400 MHz, DMSO-d₆): |
| | | | | | | 7.82 (d, 1H), 7.40 (d, 1H), 3.58 |
| | | | | | | (s, 3H), 2.33 (m, 2H), 2.23 (s, |
| | | | | | | 3H), 0.99 (dt, 3H) |
| I-1-a-7 | Me | 4-F | 5-Br | Me | Me | ¹H-NMR (400 MHz, DMSO-d₆): |
| | | | | | | 7.52 (d, 1H), 7.35 (t, 1H), 7.23 (t, |
| | | | | | | 1H), 3.58 (s, 3H), 2.33 (m, 2H), |
| | | | | | | 2.23 (s, 3H), 2.18 (s, 3H) |

3. Preparation of 4-(2-bromo-4-chloro-6-methylphenyl)-2,6-dimethyl-5-isopropoxy-3(2H)-pyridazinone (No. 1 of Table 32) 0.15 g (0.44 mmol) of the compound I-1-a-1 according to the invention of Table 31 and 0.06 g of triethylamine (1.3 eq) were initially charged in 10 ml of CH2Cl2. 0.023 g (1.0 eq) of isobutyryl chloride dissolved in 2 ml of CH2Cl2 was then added dropwise over a period of 10 min, and the mixture was stirred at room temperature for 1 h. 10 ml of five percent strength sodium bicarbonate solution were added, and the mixture was stirred for another 30 min. The organic phase was removed and dried giving, after purification by chromatography (silica gel, gradient ethyl acetate/n-heptane), 0.1 g of the target compound I-1-b-1 (yield 56.5% of theory).

1 H-NMR (400 MHz, DMSO-d6): 7.71 (d, 1H), 7.47 (d, 1H), 3.71 (s, 3H), 2.63 (m, 1H), 2.20 (s, 3H), 2.07 (s, 3H), 0.88 (dd, 6H).

The compounds of Table 32 can be obtained analogously to the method given above.

TABLE 32
Compounds of the formula (I) according to the invention in
which G is C(═O)R¹.
(I-b)

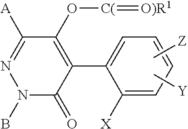

| No. | X | Y | Z | R¹ | A | B | Analytical data |
|---|---|---|---|---|---|---|---|
| I-1-b-1 | Me | 6-Br | 4-Cl | i-Pr | Me | Me | ¹H-NMR (400 MHz, |
| | | | | | | | DMSO-d₆): 7.71 (d, 1H), |
| | | | | | | | 7.47 (d, 1H), 3.71 (s, 3H), |
| | | | | | | | 2.63 (m, 1H), 2.20 (s, 3H), |
| | | | | | | | 2.07 (s, 3H), 0.88 (dd, 6H) |
| I-1-b-2 | Et | 6-Br | 4-Cl | i-Pr | Me | Me | ¹H-NMR (400 MHz, |
| | | | | | | | DMSO-d₆): 7.72 (d, 1H), |
| | | | | | | | 7.48 (d, 1H), 3.71 (s, 3H), |
| | | | | | | | 2.61 (m, 1H), 2.32, (q, |
| | | | | | | | 2H), 2.20 (s, 3H), 1.04 |
| | | | | | | | (t, 3H), 0.87 (dd, 6H) |
| I-1-b-3 | Et | 6-Cl | 4-I | Me | Me | Me | ¹H-NMR (400 MHz, |
| | | | | | | | DMSO-d₆): 7.81 (d, 1H), |
| | | | | | | | 7.68 (d, 1H), 3.71 (s, 3H), |
| | | | | | | | 2.61 (m, 1H), 2.32, (q, 2H), |
| | | | | | | | 2.22 (s, 3H), 2.07 (s, 3H) |

### 4. Preparation of 4-(2-bromo-4-chloro-6-methylphenyl)-2,6-dimethyl-5-ethoxycarbonyloxy-3(2H)-pyridazinone (No. 1 of Table 33)

0.2 g (0.42 mmol) of the compound I-1-a-1 according to the invention of Table 30 was initially charged in 20 ml of CH2Cl2, and 0.054 g of triethylamine (1.3 eq) and 0.044 g of ethyl chloroformate were added. The mixture was stirred at RT for 2 h, and 10 ml of five percent strength sodium bicarbonate solution were then added. The organic phase was separated off and then dried, concentrated and purified by column chromatography (silica gel, gradient ethyl acetate/n-heptane). This gave 0.161 g.

1H-NMR (400 MHz, DMSO-d6): 7.71 (d, 1H), 7.47 (d, 1H), 4.13 (q, 2H), 3.71 (s, 3H), 2.25 (s, 3H), 2.08 (s, 3H), 1.08 (t, 3H).

The compounds of Table 33 can be obtained analogously to the methods given above.

TABLE 33
Compounds of the formula(I) according to the invention in
which G is C(═L)MR².
(I-c)

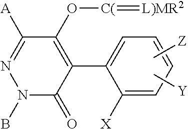

| No. | X | Y | Z | A | B | L | M | R² | Analytical data |
|---|---|---|---|---|---|---|---|---|---|
| I-1-c-1 | Me | 4-Cl | 6-Br | Me | Me | O | O | Et | ¹H-NMR (400 MHz, |
| | | | | | | | | | DMSO-d₆): 7.71 |
| | | | | | | | | | (d, 1H), 7.47 (d, 1H), |
| | | | | | | | | | 4.13 (q, 2H), 3.71 (s, |
| | | | | | | | | | 3H), 2.25 (s, 3H), |
| | | | | | | | | | 2.08 (s, 3H), 1.08 |
| | | | | | | | | | (t, 3H) |
| I-1-c-2 | Et | 4-Br | 6-Cl | Me | Me | O | O | Et | ¹H-NMR (400 MHz, |
| | | | | | | | | | DMSO-d₆): 7.71 |
| | | | | | | | | | (d, 1H), 7.57 (d, 1H), |
| | | | | | | | | | 4.12 (q, 2H), 3.71 (s, |
| | | | | | | | | | 3H), 2.33 (q, 2H), |
| | | | | | | | | | 2.23 (s, 3H), 1.08 |
| | | | | | | | | | (t, 3H), 1.04 (t, 3H) |
| I-1-c-3 | Et | 4-Cl | 6-Br | Me | Me | O | O | Et | ¹H-NMR (400 MHz, |
| | | | | | | | | | DMSO-d₆): 7.72 |
| | | | | | | | | | (d, 1H), 7.48 (d, 1H), |
| | | | | | | | | | 4.12 (q, 2H), 3.71 (s, |
| | | | | | | | | | 3H), 2.37 (m, 2H), |
| | | | | | | | | | 2.28 (s, 3H), 1.09 |
| | | | | | | | | | (t, 3H), 1.02 (t, 3H) |
| I-1-c-4 | Et | 4-Cl | 6-I | Me | Me | O | O | Et | ¹H-NMR (400 MHz, |
| | | | | | | | | | DMSO-d₆): 7.78 |
| | | | | | | | | | (d, 1H), 7.47 (d, 1H), |
| | | | | | | | | | 4.13 (q, 2H), 3.73 (s, |
| | | | | | | | | | 3H), 2.35 (m, 2H), |
| | | | | | | | | | 2.25 (s, 3H), 1.12 |
| | | | | | | | | | (t, 3H), 1.04 (t, 3H) |

### 5. Preparation of 4-(2-bromo-4-chloro-6-ethylphenyl)-2,6-dimethyl-5-methylsulfonyloxy-3(2H)-pyridazinone (No. 1 of Table 34)

0.15 g (0.41 mmol) of the compound I-1-a-3 according to the invention of Table 31 was initially charged in 10 ml of CH2Cl2, and 0.055 g of triethylamine (1.3 eq) and 0.048 g of methanesulfonyl chloride were added. The mixture was stirred at RT for 3 h, and 10 ml of five percent strength sodium bicarbonate solution were then added. The organic phase was separated off and then dried, concentrated and purified by column chromatography (silica gel, gradient ethyl acetate/n-heptane). This gave 0.145 g.

1H-NMR (400 MHz, DMSO-d6): 7.73 (d, 1H), 7.50 (d, 1H), 4.13 (q, 2H), 3.71 (s, 3H), 3.07 (s, 3H), 2.40 (m, 2H), 2.38 (s, 3H), 1.09 (t, 3H).

The compounds of Table 34 can be obtained analogously to the methods given above.

TABLE 34
Compounds of the formula (I) according to the invention in
which G is SO2R3.
(I-d)

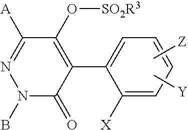

| No. | X | Y | Z | A | B | R³ | Analytical data |
|---|---|---|---|---|---|---|---|
| I-1-d-1 | Et | 4-Cl | 6-Br | Me | Me | Me | ¹H-NMR (400 MHz, |
| | | | | | | | DMSO-d₆): 7.73 (d, 1H), |
| | | | | | | | 7.50 (d, 1H), 4.13 (q, |
| | | | | | | | 2H), 3.71 (s, 3H), 3.07 |
| | | | | | | | (s, 3H), 2.40 (m, 2H), |
| | | | | | | | 2.38 (s, 3H), 1.09 (t, 3H) |

### 6. Preparation of the sodium salt of 4-(2-bromo-4-chloro-6-methylphenyl)-5-hydroxy-2,6-dimethyl-3(2H)-pyridazinone (No. 1 of Table 35)

0.1 g (0.12 mmol) of the compound I-1-a-1 according to the invention of Table 31 and 0.011 g of sodium hydroxide were dissolved in 10 ml of anhydrous methanol, and the solution was stirred for 1 hour. The mixture was concentrated under reduced pressure and taken up in toluene. The solvent was removed again, giving an amorphous powder.

The compounds of Table 35 can be obtained analogously to the methods given above.

TABLE 35
Compounds of the formula (I) according to the invention in
which G is E.
(I-g)

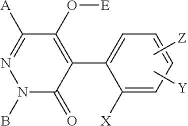

| No. | X | Y | Z | E | A | B | Analytical data |
|---|---|---|---|---|---|---|---|
| I-1-g-1 | Br | 6-Me | 4-Cl | Na⁺ | Me | Me | ¹H-NMR (400 MHz, |
| | | | | | | | DMSO-d₆): 7.38 (d, |
| | | | | | | | 1H), 7.18 (d, 1H), 3.38 |
| | | | | | | | (s, 3H), 2.05 (s, 3H), |
| | | | | | | | 1.94 (s, 3H) |

### B. Formulation Examples

a) A dust is obtained by mixing 10 parts by weight of a compound of the formula (I) and/or a salt thereof and 90 parts by weight of talc as inert substance and comminuting the mixture in a hammer mill.
b) A wettable powder which is readily dispersible in water is obtained by mixing 25 parts by weight of a compound of the formula (I) and/or a salt thereof, 64 parts by weight of kaolin-containing quartz as inert substance, 10 parts by weight of potassium lignosulfonate and 1 part by weight of sodium oleoylmethyltaurinate as wetting agent and dispersant, and grinding the mixture in a pinned-disk mill.
c) A readily water-dispersible dispersion concentrate is obtained by mixing 20 parts by weight of a compound of the formula (I) and/or a salt thereof with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to above 277° C.) and grinding the mixture in a ball mill to a fineness of below 5 microns.
d) An emulsifiable concentrate is obtained from 15 parts by weight of a compound of the formula (I) and/or a salt thereof, 75 parts by weight of cyclohexanone as solvent and 10 parts by weight of oxethylated nonylphenol as emulsifier.
e) Water-dispersible granules are obtained by mixing
   75 parts by weight of a compound of the formula (I) and/or a salt thereof,
   10 parts by weight of calcium lignosulfonate,
   5 parts by weight of sodium lauryl sulfate,
   3 parts by weight of polyvinyl alcohol and
   7 parts by weight of kaolin,
   grinding the mixture in a pinned-disk mill, and granulating the powder in a fluidized bed by spraying on water as granulating liquid.
f) Water-dispersible granules are also obtained by homogenizing and precomminuting, in a colloid mill,
   25 parts by weight of a compound of the formula (I) and/or a salt thereof,
   5 parts by weight of sodium 2,2′-dinaphthylmethane-6,6′-disulfonate,
   2 parts by weight of sodium oleoylmethyltaurinate,
   1 part by weight of polyvinyl alcohol,
   17 parts by weight of calcium carbonate and
   50 parts by weight of water,
   subsequently grinding the mixture in a bead mill and atomizing and drying the resulting suspension in a spray tower by means of a single-substance nozzle.

### C. Biological Examples

1. Pre-Emergence Herbicidal Action Against Harmful Plants

Seeds of monocotyledonous or dicotyledonous weed plants or crop plants are placed in wood-fiber pots in sandy loam and covered with soil. The compounds according to the invention, formulated in the form of wettable powders (WP) or as emulsion concentrates (EC), are then applied as aqueous suspension or emulsion at a water application rate of 600 to 800 l/ha (converted) with the addition of 0.2% of wetting agent to the surface of the covering soil. After the treatment, the pots are placed in a greenhouse and kept under good growth conditions for the test plants. The visual assessment of the damage to the test plants is carried out after a trial period of 3 weeks by comparison with untreated controls (herbicidal activity in percent (%): 100% activity=the plants have died, 0% activity=like control plants). Here, for example, the compounds Nos. I-1-b-2 (Table 32) and I-1-c-2 (Table 33) each show, at an application rate of 320 g/ha, an activity of at least 90% against Amaranthus retroflexus and Stellaria media. The compounds Nos. I-1-a-3 (Table 31) and I-1-c-3 (Table 33) each show, at an application rate of 320 g/ha, an activity of at least 80% against Lolium multiflorum and Stellaria media. The compounds Nos. I-1-c-2 (Table 33) and I-1-b-2 (Table 32) each show, at an application rate of 320 g/ha, an activity of at least 80% against Echinocloa crus galli and Lolium multiflorum.

2. Post-Emergence Herbicidal Action Against Harmful Plants

Seeds of monocotyledonous and dicotyledonous weed and crop plants are placed in sandy loam in wood-fiber pots, covered with soil and cultivated in a greenhouse under good growth conditions. 2 to 3 weeks after sowing, the test plants are treated at the one-leaf stage. The compounds according to the invention, formulated in the form of wettable powders (WP) or as emulsion concentrates (EC), are then sprayed as aqueous suspension or emulsion at a water application rate of 600 to 800 l/ha (converted) with the addition of 0.2% of wetting agent onto the green parts of the plants. After the test plants have been kept in the greenhouse under optimum growth conditions for about 3 weeks, the activity of the preparations is rated visually in comparison to untreated controls (herbicidal activity in percent (%): 100% activity=the plants have died, 0% activity=like control plants). Here, for example, the compounds Nos. I-1-c-2 and I-1-c-3 (Table 33 in each case) each show, at an application rate of 320 g/ha, an activity of at least 90% against Alopecurus myosuroides, Amaranthus retroflexus, Matricaria inodora, Pharbitis purpureum and Viola tricolor.

The compounds Nos. I-1-a-3 (Table 31) and I-1-b-2 (Table 32) each show, at an application rate of 320 g/ha, an activity of 100% against Abutilon theophrasti, Lolium multiflorum, Fallopia convolvulus and Veronica persica.

1. A 4-phenylpyridazinone of formula (I) and/or a salt thereof 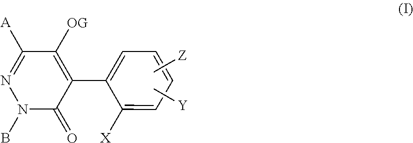
in which
A is hydrogen or (C₁-C₆)-alkyl;
B is hydrogen, (C₁-C₆)-alkyl or (C₁-C₆-alkoxy)-C₁-C₆-alkyl;
n is 0, 1, 2 or 3;
G is hydrogen, C(═O)R¹, C(=L)MR², SO₂R³, P(=L)R⁴R⁵, C(=L)NR⁶R⁷ or E;
E is a metal ion equivalent or an ammonium ion;
L is oxygen or sulfur;
M is oxygen or sulfur;
R¹ is (C₁-C₆)-alkyl, (C₂-C₆)-alkenyl, (C₁-C₄)-alkoxy-(C₁-C₆)-alkyl, di-(C₁-C₄)-alkoxy-(C₁-C₆)-alkyl or (C₁-C₄)-alkylthio-(C₁-C₆)-alkyl, each of which is substituted by n halogen atoms,
a fully saturated 3- to 6-membered ring consisting of 3 to 5 carbon atoms and 1 to 3 heteroatoms from the group consisting of oxygen, sulfur and nitrogen, which ring is substituted by n radicals from the group consisting of halogen, (C₁-C₄)-alkyl and (C₁-C₄)-alkoxy,
phenyl, phenyl-(C₁-C₄)-alkyl, heteroaryl, phenoxy-(C₁-C₄)-alkyl, heteroaryloxy-(C₁-C₄)-alkyl or (C₃-C₆)-cycloalkyl substituted by n radicals from the group consisting of halogen, (C₁-C₄)-alkyl and (C₁-C₄)-alkoxy;
R² is (C₁-C₆)-alkyl, (C₂-C₆)-alkenyl, (C₁-C₄)-alkoxy-(C₁-C₆)-alkyl or di-(C₁-C₄)-alkoxy-(C₁-C₆)-alkyl, each of which is substituted by n halogen atoms,
or (C₃-C₆)-cycloalkyl, phenyl or benzyl, each of which is substituted by n radicals from the group consisting of halogen, (C₁-C₄)-alkyl and (C₁-C₄)-alkoxy;
R³, R⁴ and R⁵ are independently of one another (C₁-C₆)-alkyl, (C₁-C₄)-alkoxy, N—(C₁-C₆)-alkylamino, N,N-di-(C₁-C₆)-alkylamino, (C₁-C₄)-alkylthio, (C₂-C₄)-alkenyl or (C₃-C₆)-cycloalkylthio, each of which is substituted by n halogen atoms,
or benzyl, phenoxy, phenylthio or phenyl substituted by n radicals from the group consisting of halogen, (C₁-C₄)-alkyl and (C₁-C₄)-alkoxy;
R⁶ and R⁷ independently of one another are each hydrogen, (C₃-C₆)-cycloalkyl, (C₂-C₆)-alkenyl, (C₁-C₆)-alkoxy, (C₁-C₄)-alkoxy-(C₁-C₆)-alkyl or (C₁-C₆)-alkyl substituted by n halogen atoms,
phenyl or benzyl, each of which is substituted by n radicals from the group consisting of halogen, (C₁-C₄)-alkyl and (C₁-C₄)-alkoxy;
or R⁶ and R⁷ together with the nitrogen atom to which they are attached form a 3- to 6-membered ring comprising 2 to 5 carbon atoms and 0 or 1 oxygen or sulfur atoms;
X is (C₁-C₆)-alkyl or (C₁-C₆)-alkyloxy;
Y is halogen;
Z is halogen, and
with the proviso that Y and Z are not both chlorine located in the 2- and 4-position.

2. The 4-phenylpyridazinone and/or salt as claimed in claim 1 in which
A is hydrogen or (C₁-C₆)-alkyl;
B is hydrogen or (C₁-C₆)-alkyl;
n is 0, 1, 2 or 3;
G is hydrogen, C(═O)R¹, C(=L)MR², SO₂R³, P(=L)R⁴R⁵, C(=L)NR⁶R⁷ or E;
E is Na⁺, K⁺, (Mg²⁺)_{1/2}, (Ca²⁺)_{1/2}, R¹³R¹⁴R¹⁵R¹⁶N⁺ or NH₄⁺;
R¹³, R¹⁴, R¹⁵ and R¹⁶ independently of one another are (C₁-C₆)-alkyl or benzyl;
L is oxygen;
M is oxygen;
R¹ is (C₁-C₆)-alkyl substituted by n halogen atoms or (C₃-C₆)-cycloalkyl, phenyl or phenyl-(C₁-C₄)-alkyl, each of which is substituted by n radicals from the group consisting of halogen, (C₁-C₄)-alkyl and (C₁-C₄)-alkoxy;
R² is (C₁-C₆)-alkyl substituted by n halogen atoms or (C₃-C₆)-cycloalkyl, phenyl or benzyl, each of which is substituted by n radicals from the group consisting of halogen, (C₁-C₄)-alkyl and (C₁-C₄)-alkoxy;
R³, R⁴ and R⁵ are each independently of one another (C₁-C₆)-alkyl substituted by n halogen atoms or phenyl or benzyl, each of which is substituted by n radicals from the group consisting of halogen, (C₁-C₄)-alkyl and (C₁-C₄)-alkoxy;
R⁶ and R⁷ are each independently of one another hydrogen, (C₁-C₆)-alkyl substituted by n halogen atoms or benzyl or phenyl substituted by n radicals from the group consisting of halogen, (C₁-C₄)-alkyl and (C₁-C₄)-alkoxy;
X is methyl, ethyl or methoxy;
Y is halogen; and
Z is halogen.

3. The 4-phenylpyridazinone and/or salt as claimed in claim 1 in which
A is hydrogen, methyl, ethyl;
B is methyl or ethyl;
n is 0, 1, 2 or 3;
G is hydrogen, C(═O)R¹, C(=L)MR², SO₂R³, P(=L)R⁴R⁵, C(=L)NR⁶R⁷ or E;
E is Na⁺, K⁺, (Mg²⁺)_{1/2}, (Ca²⁺)_{1/2}, (CH₃)₄N⁺ or NH₄⁺;
L is oxygen;
M is oxygen;
R¹ is (C₁-C₆)-alkyl or (C₃-C₆)-cycloalkyl;
R² is (C₁-C₆)-alkyl or (C₃-C₆)-cycloalkyl;
R³, R⁴ and R⁵ are each independently of one another (C₁-C₆)-alkyl, phenyl or benzyl;
R⁶ and R⁷ are each independently of one another hydrogen, (C₁-C₆)-alkyl, phenyl or benzyl;
X is methyl or ethyl;
Y is fluorine, chlorine, bromine or iodine, and
Z is fluorine, bromine or iodine.

4. A herbicidal composition comprising a herbicidally effective amount of at least one 4-phenylpyridazinone of the formula (I) and/or salt as claimed in claim 1.

5. The herbicidal composition as claimed in claim 4 comprising a mixture with at least one formulation auxiliary.

6. The herbicidal composition as claimed in claim 4 comprising at least one further pesticidally active compound selected from the group consisting of the insecticides, acaricides, herbicides, fungicides, safeners and growth regulators.

7. The herbicidal composition as claimed in claim 6, comprising a safener.

8. The herbicidal composition as claimed in claim 6 comprising a further herbicide.

9. An insecticidal composition which comprises an insecticidally effective amount of at least one 4-phenylpyridazinone of the formula (I) and/or salt as claimed in claim 1.

10. A method for controlling unwanted plants which comprises applying an effective amount of at least one 4-phenylpyridazinone of the formula (I) and/or salt as claimed in claim 1 to a plant and/or to a site of unwanted vegetation.

11. A composition for controlling unwanted plants comprising a 4-phenylpyridazinone of the formula (I) and/or salt thereof as claimed in claim 1.

12. The composition as claimed in claim 11, wherein the 4-phenylpyridazinone of the formula (I) and/or salt is suitable for controlling unwanted plants in crops of useful plants.

13. The composition as claimed in claim 12, wherein the useful plants are transgenic useful plants.

14. An insecticide comprising a 4-phenylpyridazinone of the formula (I) and/or salt as claimed in claim 1.

15. An insecticide comprising a 4-phenylpyridazinone and/or salt of claim 2.

16. An insecticide comprising a 4-phenylpyridazinone and/or salt of claim 3.

17. A method for controlling unwanted plants which comprises applying an effective amount of at least one 4-phenylpyridazinone of the formula (I) and/or salt as claimed in claim 2 to a plant and/or to a site of unwanted vegetation.

18. A method for controlling unwanted plants which comprises applying an effective amount of at least one 4-phenylpyridazinone of the formula (I) and/or salt as claimed in claim 3 to a plant and/or to a site of unwanted vegetation.
